(19) United States
(12) Patent Application Publication
(10) Pub. No.: US 2015/0338392 A1
(43) Pub. Date: Nov. 26, 2015

(54) Sterile and instantly dissolvable drug membrane and method thereof for testing drug sensitivity of antineoplastic drugs

(71) Applicant: SUZHOU MACWELL BIOMEDICA SCI & TECH CO., LTD, Suzhou, Jiangsu (CN)

(72) Inventors: Shouli Wang, Suzhou, Jiangsu (CN); Wenrong Qi, Suzhou, Jiangsu (CN); Renbo Song, Suzhou, Jiangsu (CN); Zhengyu Tao, Suzhou, Jiangsu (CN); Gang Deng, Suzhou, Jiangsu (CN); Ruibao Qiu, Suzhou, Jiangsu (CN); Minghui Wu, Suzhou, Jiangsu (CN)

(73) Assignee: SUZHOU MACWELL BIOMEDICAL SCI & TECH CO., LTD., Suzhou, Jiangsu (CN)

(21) Appl. No.: 14/759,035

(22) PCT Filed: Jan. 2, 2014
(86) PCT No.: PCT/CN2014/000001

§ 371 Date: Jul. 2, 2015

(30) Foreign Application Priority Data
Jan. 9, 2013 (CN) 201310006751.X

(51) Int. Cl. G01N 33/50

(52) U.S. Cl. CPC G01N 33/5011

(57) ABSTRACT

A sterile and instantly dissolvable drug membrane, a method thereof for testing drug sensitivity of antineoplastic drugs, and an application thereon in testing drug sensitivity of antineoplastic drugs are provided, wherein the method for testing drug sensitivity of antineoplastic drugs includes steps of: dissolving an antineoplastic drug in a cell culture fluid comprising tumor cells, in such a manner that the antineoplastic drug fully contacts with the tumor cells; wherein the antineoplastic drug is prepared into a sterile and instantly dissolvable drug membrane for being dissolved in the cell culture fluid. According to the present invention, the antineoplastic drug is prepared into a sterile and instantly dissolvable drug membrane for testing drug sensitivity of the antineoplastic drugs, which improves operation efficiency, flexibility, accuracy and economy of drug sensitivity test of the antineoplastic drugs.

### CROSS REFERENCE OF RELATED APPLICATION

This is a U.S. National Stage under 35 U.S.C 371 of the International

Application PCT/CN2014/000001, filed Jan. 2, 2014, which claims priority under 35 U.S.C. 119(a-d) to CN 201310006751.X, filed Jan. 9, 2013.

### BACKGROUND OF THE PRESENT INVENTION

1. Field of Invention

The present invention relates to a sterile and instantly dissolvable drug membrane, a method thereof for testing drug sensitivity of antineoplastic drugs, and an application thereof for testing drug sensitivity of antineoplastic drugs, which belong to a field of medical technology.

2. Description of Related Arts

Cancer is a common disease which seriously harms human life and health, and is a major cause of disability and premature death. For an age group from 35 to 59, cancer ranks a first cause of death. Statistics show that the number of cancer cases in China is annually 2 million, wherein 1.5 million thereof end up with death. Moreover, the number increases by 3% per year and patients tend to be younger. Cancer is the most deadly in all kinds of diseases.

Conventionally, main treatment methods for cancer comprise surgery, radiotherapy, chemotherapy, hormonal therapy and immune therapy, wherein compared with other methods, chemotherapy, as a systemic treatment, is able to kill tumor cells as many as possible. Therefore, chemotherapy plays a very important role in the treatment of cancer. With the development of medicine, chemotherapy is no longer simply a method of palliative treatment, and is changing from palliative to curative.

In 1998, the World Health Organization announced that appropriate chemotherapy had become a curing method for some tumors (such as malignant trophoblastic tumor, acute lymphocytic leukemia, Hodgkin's lymphoma, non-Hodgkin's lymphoma, testicular cancer, acute myeloid leukemia, embryonal rhabdomyosarcoma, skin cancer, small-cell lung cancer and ovarian cancer) has become the radical treatment can cure cancer. With the help of chemotherapy, tumors such as mammary cancer, osteogenic sarcoma, colon cancer, osteosarcoma, retinoblastoma, soft tissue sarcoma and renal blastoma are curable.

Although chemotherapy plays a very important role in the treatment of cancer, the results are often unsatisfactory in clinical practice. Tumor cells will become resistant to chemotherapy drugs, which is a common factor leading to failure of cancer chemotherapy, and is a key problem troubling cancer treatment. Drug resistance is a very common clinical problem. According to the American Cancer Society, more than 90% of patients died due to tumor were affected by drug resistance by varying degrees.

Tumor cell drug resistance is divided into primary resistance and acquired resistance. Conventional, clinical practice is generally based on evidence-based research results of international tumor clinical experiments, for obtaining that different chemotherapeutic drugs have different sensitivity to different tumor treatments, which means that each tumor has a corresponding effective chemotherapeutic drug sensitivity profile. Accordingly, a drug list with highest efficacy or pharmaceutical composition treatment is selected for treatment. However, in clinical, an effective treatment recognized by the evidence-based cancer research for certain tumor has no effect on some patients. For example, doxorubicin for invasive breast cancer is a landmark of treatment, but there are still 50% of invasive breast cancer patients who are not sensitive to the drug.

Again, Gemzar is significantly effective for non-small cell lung cancer, but there are more than 60% of the patients receive no obvious effect from Gemzar.

Tumors have significant individual differences for various chemotherapeutic drugs. That is to say, for patients of different tumor types or different patients of the same type, or even the same patient at different stages, the sensitivity to chemotherapy is quite different, and treatment effects varies widely. So far there is not a chemotherapy drug or combination of several chemotherapy drugs, is 100% effective for a particular tumor. Clinically, it is apparently unreasonable to use the same chemotherapy drug or chemotherapy method on different tumor patients. Therefore, it is quite necessary to provide tumor chemotherapy sensitivity test on different patients for selecting effective drugs. For the propose, testing similar to bacterial sensitivity testing is necessary, for accurately screening chemotherapy drug according to different patients and determining the dosage with a reliable method, so as to truly personalize medicine clinic.

Conventionally, drugs for tumor drug sensitivity testing are mainly prepared right before utilization. Tumor drug sensitivity test needs to test a variety of antineoplastic drugs. Drug liquid is complex to prepare and dilute, and a dosage required is small, which is inconvenient to sample. Some of the drugs in a solution state are only able to be stored for a short time, and the remaining part must be wasted, resulting in waste of drugs, especially for expensive drugs.

The prior art discloses a tumor drug sensitivity testing methods, mainly using reagent kits. For example, Chinese patent CN93111551.5 discloses a tumor chemotherapy drug sensitivity prediction method and reagent kits thereof. According to the patent, 8 kinds of commonly used antineoplastic drugs are pre-set in specimen bottles with pre-determined dosages. However, the method is not suitable for testing combined chemotherapy, and is not suitable for adjusting dosages thereof

Chinese patent CN03102260.X uses a pre-prepared drug sensitivity testing plate, adding antineoplastic drugs onto the plate with a pre-calculated dosage, freeze-dries and packages for cancer drug sensitivity testing. The method is also not suitable for testing combined chemotherapy, and is not suitable for adjusting dosages thereof. Furthermore, the method is not really sterile, which does not meet the requirements of sterile cell culture. Because the dosage of the drug added onto the plates is small, intersect dust pollution of different drugs will happen during freeze-drying. Therefore, the actual diagnosis is suspicious.

Clinically, there are a few antineoplastic drug sensitivity testing methods whose antineoplastic drugs are prepared right before utilization. However, the methods have many problems: the exact concentration of the drug is unknown, the accuracy of the drug liquid is doubtable; drugs for preparation are clinical ones whose excipients may interfere with the final result of judgment; antineoplastic drug sensitivity testing is not applicable to clinical oral drugs; and individual operator error is large.

### SUMMARY OF THE PRESENT INVENTION

In order to overcome conventional defects in drug sensitivity test of antineoplastic drugs, the present invention provides a method of improving operation efficiency, flexibility, accuracy and economy of drug sensitivity test of antineoplastic drugs. The technical solution of the present invention is as follows.

The present invention provides a method for testing drug sensitivity of antineoplastic drugs, comprising steps of: dissolving an antineoplastic drug in a cell culture fluid comprising tumor cells, in such a manner that the antineoplastic drug fully contacts with the tumor cells; wherein the antineoplastic drug is prepared into a sterile and instantly dissolvable drug membrane for being dissolved in the cell culture fluid. The cell culture fluid is not particularly limited, and may be a conventional cell culture fluid such as RPMI1640, DMEM, 199, MEM, F12, and L-15.

Accordingly, the sterile and instantly dissolvable drug membrane prepared with the antineoplastic drug is easy to dissolve in the cell culture fluid. A thickness and an area thereof are reasonably controlled, for ensuring dissolving speed and mechanical strength, wherein the thickness of the sterile and instantly dissolvable drug membrane is 0.01 mm-1 mm, preferably 0.04-0.15 mm; the area thereof is 0.2 cm²-25 cm², preferably 0.5 cm²-16² cm; wherein 1 cm² of the sterile and instantly dissolvable drug membrane is dissolved in 1 ml of cell culture fluid within 5 min, preferably 3 min, more preferably 100 sec.

Accordingly, the sterile and instantly dissolvable drug membrane comprises the antineoplastic drug with an amount of 0.0001 wt %-30 wt % , a membrane forming material with an amount of 50 wt %-98 wt %, and a plasticizer with an amount of 1 wt %-20 wt %;

wherein the membrane forming material is polyvinyl alcohol, hydroxypropyl methyl cellulose, hydroxyethyl propyl cellulose, sodium carboxymethyl cellulose, or polyvinylpyrrolidone; preferably polyvinyl alcohol, especially polyvinyl alcohol 04-88 or polyvinyl alcohol 05-88;

wherein the membrane forming material is water-dissolvable material; in view of manufacturing difficulty and forming performance, the membrane forming material is preferably polyvinyl alcohol, hydroxypropyl methyl cellulose, and polyvinylpyrrolidone;

in view of dissolubility, mechanical performance, etc., the membrane forming material is preferably polyvinyl alcohol, especially polyvinyl alcohol 04-88 and polyvinyl alcohol 05-88.

With increase of polymerization, water-dissolubility of the polyvinyl alcohol decreases, and strength thereof after membrane forming increases. Water-dissolubility of polyvinyl alcohol 04-88 and polyvinyl alcohol 05-88 is relatively sufficient, and strength thereof after membrane forming is enough. For drugs which have low contents in the membrane and hardly affects membrane strength, the polyvinyl alcohol 04-88 is sufficient. For drugs which have high contents in the membrane and greatly affects membrane strength, the polyvinyl alcohol 05-88 is used to partly or entirely replace the polyvinyl alcohol 04-88, so as to improve membrane strength.

The plasticizer is glycerol, polyethylene glycol, ethylene glycol, propylene glycol, sorbitol, or triethyl citrate; preferably glycerol.

Accordingly, the antineoplastic drug is not particularly limited, which may be any of the conventional antineoplastic drugs. For example, the antineoplastic drug is selected from a group consisting of fluorouracil, tegafur, tegadifur, doxifluridine, carmo fur, methotrexate, busulfan, doxorubicin, daunorubicin, idarubicin, epirubicin, pirarubicin, mitoxantrone, actinomycin D, bleomycin, pingyangmycin, mitomycin, mithramycin, olivomycin, streptozocin, mechlorethamine, chlorambucil, melphalan, cyclophosphamide, ifosfamide, thiotepa, carmustine, lomustine, cytarabine, floxuridine, cyclocylidine, chlorine cyclocylidine, fludarabine, gemcitabine, docetaxel, vinorelbine, vindesine, vincristine, paclitaxel, camptothecin, hydroxy camptothecin, cisplatin, oxaliplatin, carboplatin, nedaplatin, lobaplatin, platinum heptyl, rituximab, ibritumomab, cetuximab, bevacizumab, interleukin-2, leuprorelin acetate, goserelin acetate, anastrozole, letrozole, aminoglutethimide, formestane, exemestane, teniposide, etoposide, pentostatin, irinotecan, sorafenib, capecitabine, decitabine, pemetrexed disodium, procarbazine, harringtonine, thalidomide, dacarbazine, tamoxifen, temozolomide, topotecan, gefitinib, erlotinib, flutamide, nilutamide, bicalutamide, exemestane, arsenite and indirubin.

The cell culture fluid of testing is a liquid culture medium for culturing tumor cells, which comprises amino acid, glucose, inorganic salts, vitamins and trace elements. For stabilizing a pH value, there is generally a pH buff system in the culture fluid, while a small amount of phenol red is added as a pH indicator. Additionally, a certain amount of serum or growth-promoting factors are generally added into the culture fluid. A suitable tumor cell culture fluid may be RPMI1640 (with 10%-20% fetal bovine serum and/or 0.03% glutamine), DMEM, 199, MEM, F12, and L-15; most usually RPMI 1640 with 10%-20% fetal bovine serum. The tumor cells are different from other cells. Therefore, growth-promoting factors are needed during culturing, which is illustrated in Table 1.

TABLE 1
common growth-promoting factors and final concentration thereof

| Additive | Final concentration | |
|---|---|---|
| BSA | 10⁻⁵ | mol/ml |
| Transferrin | 5-10 | μg/ml |
| Insulin | 5 | μg/ml |
| Hydrocortisone | 10⁻⁶ | mol/ml |
| EGF | 5 | ng/ml |
| FGF | 5 | ng/ml |
| NGF | 5 | ng/ml |

For example, HITES, which is suitable for human small cell lung cancer, is a modified RPMI 1640 culture medium, comprising hydrocortisone, insulin, transferring, estrogen, and selenium.

The tumor cells may be separated tumor tissue obtained by clinical surgical resection, or non-solid tumor cells in vitro separated from blood or body fluid. The tumor cells should be fresh, sterile, timely, and accurate. The cancer tissue should be cultured as soon as possible after be obtained. Usually, within 4 hours, cell survival rate is best. Specimen is stored at 4° C., with no more than 24 hours. It should be noted that during separating, large tumor tissue have degeneration or necrosis areas, which should be avoid; and active areas should be selected. At the same time, because solid tumor tissue may be contaminated during separating, attention should be paid to cleaning and sterilization. For fresh tumor tissue, separating with purely mechanical methods such as pipetting and filtration hardly harms cancer cells, which is suitable for some tumors such as human ovarian cancer and glioma. Cancer tissues are usually solid, wherein tumor cells are embedded in a large number of fiber base materials, and are difficult to be mechanically separated. Therefore, enzyme digestion method is often used to disperse cancer cells. Cancer cells prepared after digestion should have sufficient cell density during culturing. Usually, seeded cell concentration is usually 5×10⁵/ml or 1×10⁶/ml, and culturing time is 37° C.

Another object of the present invention is to provide a sterile and instantly dissolvable drug membrane for testing drug sensitivity of antineoplastic drugs, comprising: an antineoplastic drug with an amount of 0.0001 wt %-30 wt %, a membrane forming material with an amount of 50 wt %-98 wt %, and a plasticizer with an amount of 1 wt %-20 wt %; wherein the membrane forming material is polyvinyl alcohol, hydroxypropyl methyl cellulose, hydroxyethyl propyl cellulose, sodium carboxymethyl cellulose, or polyvinylpyrrolidone;

wherein the membrane forming material is water-dissolvable material; in view of manufacturing difficulty and forming performance, the membrane forming material is preferably polyvinyl alcohol, hydroxypropyl methyl cellulose, and polyvinylpyrrolidone.

In view of dissolubility, mechanical performance, etc., the membrane forming material is preferably polyvinyl alcohol, especially polyvinyl alcohol 04-88 and polyvinyl alcohol 05-88.

The plasticizer is glycerol, polyethylene glycol, ethylene glycol, propylene glycol, sorbitol, or triethyl citrate; preferably glycerol.

Preferably, the sterile and instantly dissolvable drug membrane comprises an antineoplastic drug with an amount of 0.0001 wt %-10 wt %, a membrane forming material with an amount of 80 wt %-98wt %, and a plasticizer with an amount of 1 wt %-10 wt %.

The present invention also provides a method for preparing a sterile and instantly dissolvable drug membrane as above, comprising steps of:

(1) adding a membrane forming material into water, and stirring or heating for dissolving;

(2) adding a plasticizer and an antineoplastic drug, stirring for thoroughly dissolving, and degassing with heating, staying, or ultrasound;

(3) coating a solution obtained above on a membrane producer, and drying with hot wind or cold wind;

(4) stripping and measuring contents thereof, dividing according to a measuring result for obtaining a pre-determined dosage; and

(5) after dividing, sterilizing with irradiation sterilization or epoxyethane sterilization, preferably the irradiation sterilization.

The sterile and instantly dissolvable drug membrane as above is suitable for testing drug sensitivity of antineoplastic drugs, wherein the sterile and instantly dissolvable drug membrane is directed used in a tumor cell drug sensitivity test, or the sterile and instantly dissolvable drug membrane is dissolved and then activated by adding drug metabolic enzyme, for being used in the tumor cell drug sensitivity test.

Drug sensitivity test of antineoplastic drugs requires that the antineoplastic drug is dissolved in the cell culture fluid, in such a manner that the drug sufficiently contacts with the cells. To ensure that operation is reliable and convenient, the drug must be able to quickly disperse and dissolve in the cell culture fluid. The present invention prepares the sterile and instantly dissolvable drug membrane with the antineoplastic drug for testing drug sensitivity thereof, which is easy to operate and saves costs. Accordingly, the antineoplastic drug is prepared into a drug membrane with exact dosage, good stability, and rapid dissolubility in tumor cell culture fluid. Furthermore, the drug membrane is accurately sliced according to dosages, which is convenient for drug sensitivity test of the antineoplastic drugs.

Dissolution Speed Test

Providing 10 blank drug membranes with an area of 1 cm² for each group, respectively adding to a 24-well plate, adding 1 ml cold RPMI 1640 cell culture fluid (comprising 20% FBS) to each well which has one of the blank drug membranes, softly shaking until the blank drug membranes are fully dissolved; wherein time is recorded and averaged, see Table 2.

TABLE 2
dissolution speed test (membrane forming
material 98%, plasticizer glycerol 2%)

| Membrane | Dissolving time (s) | | | |
|---|---|---|---|---|
| thickness | polyvinyl | polyvinyl | | polyvinyl- |
| (mm) | alcohol 04-88 | alcohol 05-88 | gelatin | pyrrolidone |
| 0.01 mm | 23 | 28 | 35 | 18 |
| 0.05 mm | 40 | 46 | 58 | 29 |
| 0.10 mm | 49 | 61 | 90 | 41 |
| 0.15 mm | 56 | 77 | 214 | 53 |
| 0.20 mm | 70 | 98 | 322 | 73 |

Stretching Performance Test

Providing 10 blank drug membranes for each group, providing stretching performance with a universal testing machine, reading and averaging stretching strength and breaking extending rate, which refer to Table 3 and Table 4.

TABLE 3
stretching strength test of blank drug membranes (membrane
forming material 98%, plasticizer glycerol 2%)

| | Stretching strength (Mpa) | | | | |
|---|---|---|---|---|---|
| | | | hydroxy- | | |
| Membrane | polyvinyl | polyvinyl | propyl- | methacrylic | |
| thickness | alcohol | alcohol | methyl | acid | |
| (mm) | 04-88 | 05-88 | cellulose | copolymer | amioca |
| 0.01 mm | 2.1 | 3.7 | 3.2 | 1.5 | 1.2 |
| 0.05 mm | 12.8 | 13.5 | 15.6 | 1.9 | 1.7 |
| 0.10 mm | 16.9 | 18.8 | 22.9 | 2.8 | 2.3 |
| 0.15 mm | 28.5 | 34.2 | 43.1 | 3.1 | 2.9 |
| 0.20 mm | 45.7 | 55.2 | 61.4 | 3.7 | 3.2 |

TABLE 4
breaking strength test of blank drug membranes (membrane
forming material 98%, plasticizer glycerol 2%)

| | Breaking extending rate (%) | | | | |
|---|---|---|---|---|---|
| | | | carboxy- | | |
| Membrane | polyvinyl | polyvinyl | methyl- | | |
| thickness | alcohol | alcohol | cellulose | propylene | polyacrylic |
| (mm) | 04-88 | 05-88 | sodium | oxide | acid |
| 0.01 mm | 0.7 | 0.9 | 0.7 | 0.2 | 0.3 |
| 0.05 mm | 3.0 | 3.8 | 3.2 | 0.9 | 1.2 |
| 0.10 mm | 4.8 | 6.1 | 6.5 | 1.3 | 1.8 |
| 0.15 mm | 5.2 | 6.7 | 7.1 | 1.6 | 2.7 |
| 0.20 mm | 5.6 | 7.0 | 8.5 | 2.5 | 3.8 |

TABLE 5
performance test of polyvinyl alcohol blank
drug membranes (with a thickness of 0.10 mm)

| | polyvinyl | polyvinyl | glyc- | | stretching | breaking |
|---|---|---|---|---|---|---|
| | alcohol | alcohol | erol | dissolving | strength | extending |
| No. | (%) | (%) | (%) | time (s) | (Mpa) | rate (%) |
| 1 | 99 | — | 1 | 55 | 19.1 | 2.3 |
| 2 | 96 | — | 4 | 46 | 14.8 | 6.2 |
| 3 | 90 | — | 10 | 38 | 10.2 | 7.9 |
| 4 | 80 | — | 20 | 29 | 6.7 | 8.1 |
| 5 | — | 99 | 1 | 66 | 22.8 | 3.5 |
| 6 | — | 96 | 4 | 56 | 16.3 | 6.8 |
| 7 | — | 90 | 10 | 47 | 12.7 | 8.1 |
| 8 | — | 80 | 20 | 40 | 9.5 | 8.5 |
| 9 | 70 | 28 | 2 | 48 | 18.5 | 5.6 |
| 10 | 49 | 49 | 2 | 51 | 19.9 | 5.1 |
| 11 | 28 | 70 | 2 | 60 | 19.2 | 6.2 |

### DETAILED DESCRIPTION OF THE PREFERRED EMBODIMENT

Preferred embodiment 1:

| | | |
|---|---|---|
| vincristine | 0.04 | g |
| polyvinyl alcohol 04-88 | 97.96 | g |
| glycerol | 2 | g |
| pure water | 200 | ml |

Heating water to 80° C., adding polyvinyl alcohol 04-88 and stirring for dissolving; cooling to 25° C., adding glycerol and vincristine, stirring for 0.5 h; stopping stirring, staying for 4 h, and removing air bubbles; coating and drying at 30° C.; stripping, cutting and packaging; then sterilizing.

Preferred embodiment 2:

| | | |
|---|---|---|
| etoposide | 1.25 | g |
| polyvinyl alcohol 05-88 | 96.75 | g |
| glycerol | 2 | g |
| pure water | 400 | ml |

Heating water to 80° C., adding polyvinyl alcohol 05-88 and stirring for dissolving; cooling to 25° C., adding glycerol and etoposide, stirring for 0.5 h; stopping stirring, staying for 4 h, and removing air bubbles; coating and drying at 30° C.; stripping, cutting and packaging; then sterilizing.

Preferred embodiment 3:

| | | |
|---|---|---|
| methotrexate | 0.4 | g |
| polyvinyl alcohol 04-88 | 97.6 | g |
| polyethylene glycol | 2 | g |
| pure water | 200 | ml |

Heating water to 80° C., adding polyvinyl alcohol 04-88 and stirring for dissolving; cooling to 25° C., adding polyethylene glycol and methotrexate, stirring for 0.5 h; stopping stirring, staying for 4 h, and removing air bubbles; coating and drying at 30° C.; stripping, cutting and packaging; then sterilizing.

Preferred embodiment 4:

| | | |
|---|---|---|
| cytarabine | 0.5 | g |
| hydroxypropyl methyl cellulose | 97.5 | g |
| glycerol | 2 | g |
| pure water | 200 | ml |

Heating water to 80° C., adding hydroxypropyl methyl cellulose and stirring for dissolving; cooling to 25° C., adding glycerol and cytarabine, stirring for 0.5 h; stopping stirring, staying for 4 h, and removing air bubbles; coating and drying at 30° C.; stripping, cutting and packaging; then sterilizing.

Preferred embodiment 5:

| | | |
|---|---|---|
| hydroxy camptothecin | 0.01 | g |
| sodium carboxymethyl cellulose | 97.99 | g |
| glycerol | 2 | g |
| pure water | 400 | ml |

Heating water to 80° C., adding sodium carboxymethyl cellulose and stirring for dissolving; cooling to 70° C., adding glycerol and hydroxy camptothecin, stirring for 0.5 h; stopping stirring, keeping temperature for 1 h, and removing air bubbles; coating and drying at 70° C.; stripping, cutting and packaging; then sterilizing.

Preferred embodiment 6:

| | | |
|---|---|---|
| homoharringtonine | 0.0012 | g |
| polyvinyl alcohol 05-88 | 97.999 | g |
| ethylene glycol | 2 | g |
| pure water | 400 | ml |

Heating water to 80° C., adding polyvinyl alcohol 05-88 and stirring for dissolving; cooling to 25° C., adding ethylene glycol and homoharringtonine, stirring for 0.5 h; stopping stirring, staying for 4 h, and removing air bubbles; coating and drying at 30° C.; stripping, cutting and packaging; then sterilizing.

Preferred embodiment 7:

| | | |
|---|---|---|
| daunorubicin | 0.008 | g |
| polyvinylpyrrolidone | 97.992 | g |
| glycerol | 2 | g |
| pure water | 200 | ml |

Heating water to 80° C., adding polyvinylpyrrolidone and stirring for dissolving; cooling to 25° C., adding glycerol and daunorubicin, stirring for 0.5 h; stopping stirring, staying for 4 h, and removing air bubbles; coating and drying at 30° C.; stripping, cutting and packaging; then sterilizing.

Preferred embodiment 8:

| | | |
|---|---|---|
| mitoxantrone | 0.005 | g |
| polyvinyl alcohol 04-88 | 97.995 | g |
| triethyl citrate | 2 | g |
| pure water | 400 | ml |

Heating water to 80° C., adding polyvinyl alcohol 04-88 and stirring for dissolving; cooling to 25° C., adding triethyl citrate and mitoxantrone, stirring for 0.5 h; stopping stirring, staying for 4 h, and removing air bubbles; coating and drying at 30° C.; stripping, cutting and packaging; then sterilizing.

Preferred embodiment 9:

| | | |
|---|---|---|
| doxorubicin | 0.2 | g |
| polyvinyl alcohol 05-88 | 97.8 | g |
| propylene glycol | 2 | g |
| pure water | 200 | ml |

Heating water to 80° C., adding polyvinyl alcohol 05-88 and stirring for dissolving; cooling to 25° C., adding propylene glycol and doxorubicin, stirring for 0.5 h; stopping stirring, staying for 4 h, and removing air bubbles; coating and drying at 30° C.; stripping, cutting and packaging; then sterilizing.

Preferred embodiment 10:

| | | |
|---|---|---|
| idarubicin | 0.05 | g |
| polyvinyl alcohol 04-88 | 97.95 | g |
| glycerol | 2 | g |
| pure water | 200 | ml |

Heating water to 80° C., adding polyvinyl alcohol 04-88 and stirring for dissolving; cooling to 25° C., adding glycerol and idarubicin, stirring for 0.5 h; stopping stirring, staying for 4 h, and removing air bubbles; coating and drying at 30° C.; stripping, cutting and packaging; then sterilizing.

Preferred embodiment 11:

| | | |
|---|---|---|
| polyvinyl alcohol 04-88 | 98 | g |
| glycerol | 2 | g |
| pure water | 200 | ml |

Heating water to 80° C., adding polyvinyl alcohol 04-88 and stirring for dissolving; cooling to 70° C., adding glycerol, stirring for 0.5 h; stopping stirring, staying for 1 h, and removing air bubbles; coating and drying at 70° C.; stripping, cutting and packaging; then sterilizing.

Preferred Embodiment 12:

Drug sensitivity of antineoplastic drugs

Selecting chemotherapy drugs according to treatment guidelines of acute myeloid leukemia, preparing drug membranes according to natures of the drugs and peak plasma concentration of the drugs in human body, and preparing blank drug membranes according to preferred embodiments 1-11; according to measuring results, cutting the drug membrane for comprising the chemotherapy drug with 1 ml peak plasma concentration, and cutting blank drug membrane into 1 cm².

Respectively acquiring 10 ml marrow (heparin) from 20 patients with acute myeloid leukemia with aseptic techniques before chemotherapy, separating leukemia cells with Ficoll in a cleaned room, washing with culture fluid and putting in RPMI 1640 culture system for obtaining 1×10⁶/ml cell suspension.

Seeding to cell suspension in a 24-well plate (with 1 ml/well), providing a cell control group (tumor cell suspension), a blank drug membrane control group (blank drug membrane+tumor cell suspension), and an experimental group (tumor cell suspension+chemotherapy drug membrane); wherein each drugs in the experimental groups are added in three wells, and no chemotherapy drug is added to the wells for the control group; the experimental group comprises a single-drug group and a combined-drug group (referring to Table 6); when using combined-drugs, combining corresponding single-drugs according to a chemotherapy regimen, and placing in a same well; adding corresponding drug membrane to each well as pre-determined, and adding the cell suspension with 1 ml/well, gently shaking until the blank drug membranes are fully dissolved; culturing at 37° C. with 5% carbon dioxide and saturated humidity for 24 hours; collecting suspension cells with centrifugation, wherein a micro-centrifuge speed is 2000 RPM, and a centrifugation time is 5 min; the culture medium is discarded; washing the cells twice with cold PBS (2000 RPM, centrifugation time 5 min for collecting cells); buffing the suspension cells with 400 μl IX Binding Buffer, wherein a concentration is about 1×10⁶/ml adding 5 μl Annexin V-FITC in the cell suspension and gently mixing, then incubating at 2-8° C. in dark for 15 min; adding 10 μl PI and gently mixing, then incubating at 2-8° C. in dark for 5 min; measuring apoptosis and necrosis of the drug control group and the experimental group within one hour by a flow cytometry, so as to calculate inhibition rate of drugs and drug sensitivity.

TABLE 6
drug sensitivity according to test

| Drug | Sensitivity (%) |
|---|---|
| vincristine membrane | 25 |
| etoposide membrane | 10 |
| methotrexate membrane | 0 |
| cytarabine membrane | 35 |
| hydroxy camptothecin membrane | 25 |
| homoharringtonine membrane | 35 |
| daunorubicin membrane | 65 |
| mitoxantrone membrane | 25 |
| doxorubicin membrane | 40 |
| idarubicin membrane | 30 |
| cytarabine membrane + daunorubicin | 95 |
| membrane | |
| cytarabine membrane + idarubicin | 70 |
| membrane | |
| cytarabine membrane + doxorubicin | 85 |
| membrane | |
| cytarabine membrane + | 85 |
| homoharringtonine membrane | |

(inhibition rate = [(active cells of blank drug membrane control group − active cells of experimental group)/active cells of blank drug membrane control group] × 100%; wherein inhibition rate >50% means sensitive)

Preferred Embodiment 13:

Selecting chemotherapy drugs according to treatment guidelines of small cell lung cancer, preparing drug membranes according to natures of the drugs and peak plasma concentration of the drugs in human body, and preparing blank drug membranes according to preferred embodiments 1-11; according to measuring results, cutting the drug membrane for comprising the chemotherapy drug with 1 ml peak plasma concentration, and cutting blank drug membrane into 1 cm².

Before chemotherapy, taking a fresh small cell lung cancer tissue block (0.5 cm³-1 cm³) from a patient, placing in sterile saline (containing 200,000 units/ml penicillin, 250,000 units/ml streptomycin), cutting off fat, fiber and other normal tissue on an ultra clean table, as well as necrosis and bloody tissues; washing with saline comprising antibiotics, adding tissue digestive enzymes (such as trypsin and collagenase) for digesting for 2-3 h, in such a manner that tissue blocks becomes individual cells and pass through a copper mesh; centrifuging for removing supernatant, adding RPMI 1640 culture medium for pipetting into cell suspension, counting and adjusting cell concentration to 1×10⁶/ml.

Seeding to cell suspension in a 24-well plate (with 1 ml/well), providing a cell control group (tumor cell suspension), a blank drug membrane control group (blank drug membrane+tumor cell suspension), and an experimental group (tumor cell suspension+chemotherapy drug membrane); wherein each drugs in the experimental groups are added in three wells, and no chemotherapy drug is added to the wells for the control group; the experimental group comprises a single-drug group and a combined-drug group; when using combined-drugs, combining corresponding single-drugs according to a chemotherapy regimen, and placing in a same well; adding corresponding drug membrane and activating enzyme to each well as pre-determined, and adding the cell suspension with 1 ml/well, gently shaking until the blank drug membranes are fully dissolved; culturing at 37° C. with 5% carbon dioxide and saturated humidity for 24 hours; collecting suspension cells with centrifugation, wherein a micro-centrifuge speed is 2000 RPM, and a centrifugation time is 5 min; the culture medium is discarded; washing the cells twice with cold PBS (2000 RPM, centrifugation time 5 min for collecting cells); buffing the suspension cells with 400 μl IX Binding Buffer, wherein a concentration is about 1×10⁶/ml; adding 5 μl Annexin V-FITC in the cell suspension and gently mixing, then incubating at 2-8° C. in dark for 15 min; adding 10 μl PI and gently mixing, then incubating at 2-8° C. in dark for 5 min; measuring apoptosis and necrosis of the drug control group and the experimental group within one hour by a flow cytometry, so as to calculate inhibition rate of drugs.

TABLE 7
chemotherapy regimens and inhibition rates
(when using combined-drugs, combining corresponding
single-drugs according to a chemotherapy regimen, and
adding corresponding corresponding activating enzyme)

| | Inhibition |
|---|---|
| Regimen | rate (%) |
| etoposide membrane | 76 |
| paclitaxel membrane | 69 |
| topotecan membrane | 61 |
| etoposide membrane + cisplatin membrane | 90 |
| etoposide membrane + carboplatin membrane | 83 |
| irinotecan membrane + cisplatin membrane | 80 |
| carboplatin membrane + paclitaxel membrane + | 96 |
| etoposide membrane | |
| cyclophosphamide membrane + doxorubicin | 85 |
| membrane + vincristine membrane + | |
| hepatomicrosome enzyme | |
| cyclophosphamide membrane + doxorubicin | 84 |
| membrane + etoposide membrane + | |
| hepatomicrosome enzyme | |

(inhibition rate = [(active cells of blank drug membrane control group − active cells of experimental group)/active cells of blank drug membrane control group] × 100%; wherein cyclophosphamide should be activated by hepatomicrosome enzyme)

Preferred Embodiment 14:

Selecting chemotherapy drugs according to treatment guidelines of mammary cancer, preparing drug membranes according to natures of the drugs and peak plasma concentration of the drugs in human body, and preparing blank drug membranes according to preferred embodiments 1-11; according to measuring results, cutting the drug membrane for comprising the chemotherapy drug with 1 ml peak plasma concentration, and cutting blank drug membrane into 1 cm².

Immersing a surgically removed mammary cancer tissue in sterile Hanks liquid, adding a few normal tissues into a RPMI 1640 culture medium comprising 1% double-antibody and washing repeatedly, cutting into 1-2mm³ blocks; adding 10 times 0.25% trypsin solution and 0.02% ethylene diamine tetraacetic acid (EDTA), digesting at 37° C. for 40min, shanking once every 5min, removing digesting liquid and adding serum-free 1640 culture medium for washing, then adding a little culture fluid and pipetting with a suction tube, in such a manner that single cells are formed and pass though a 40 mesh net; and preparing into 1×10⁶/ml suspension with RPMI 1640 culture system.

Seeding to cell suspension in a 24-well plate (with 1 ml/well), providing a cell control group (tumor cell suspension), a blank drug membrane control group (blank drug membrane+tumor cell suspension), and an experimental group (tumor cell suspension+chemotherapy drug membrane); wherein each drugs in the experimental groups are added in three wells, and no chemotherapy drug is added to the wells for the control group; when using combined-drugs, combining corresponding single-drugs according to a chemotherapy regimen, and placing in a same well; adding corresponding drug membrane and activating enzyme to each well as pre-determined, and adding the cell suspension with 1 ml/well, gently shaking until the blank drug membranes are fully dissolved; placing the 24-well plate in a 5% CO₂ incubator at 37° C., adding 20 μl MTT to each well and gently mixing, incubating for 6-12 h, taking the 24-well plate and centrifuging for 15 min with a speed of 1000 r/min, adding 100 μl DMSO into each well, thoroughly shaking until purple crystal in each well is fully dissolved; then analyzing absorbance (A) value of each well at 570 nm with an enzyme labeled analyzer.

Inhibition rates of different chemotherapy drugs on mammary cancer cells are calculated as follows:

mammary cancer cell inhibition rate=(1−A value of experimental group/A value of blank drug membrane control group)×100%.

TABLE 8
chemotherapy regimens and inhibition rates
(when using combined-drugs, combining corresponding
single-drugs according to a chemotherapy regimen, and
adding corresponding corresponding activating enzyme)

| | Inhibition |
|---|---|
| Regimen | rate (%) |
| cyclophosphamide membrane + doxorubicin membrane + | 83 |
| docetaxel membrane + hepatomicrosome enzyme | |
| cyclophosphamide membrane + doxorubicin membrane + | 76 |
| paclitaxel membrane + hepatomicrosome enzyme | |
| cyclophosphamide membrane + docetaxel membrane + | 78 |
| hepatomicrosome enzyme | |
| cyclophosphamide membrane + doxorubicin membrane + | 55 |
| hepatomicrosome enzyme | |
| cyclophosphamide membrane + doxorubicin membrane + | 71 |
| fluorouracil membrane + hepatomicrosome enzyme | |
| cyclophosphamide membrane + methotrexate membrane + | 63 |
| fluorouracil membrane + hepatomicrosome enzyme | |
| cyclophosphamide membrane + epirubicin membrane + | 79 |
| hepatomicrosome enzyme | |

(inhibition rate = [(active cells of blank drug membrane control group − active cells of experimental group)/active cells of blank drug membrane control group] × 100%).

1-10. (canceled)

11. A method for testing drug sensitivity of antineoplastic drugs, comprising steps of: dissolving an antineoplastic drug in a cell culture fluid comprising tumor cells, in such a manner that the antineoplastic drug fully contacts with the tumor cells; wherein the antineoplastic drug is prepared into a sterile and instantly dissolvable drug membrane for being dissolved in the cell culture fluid.

12. The method, as recited in claim 11, wherein a thickness of the sterile and instantly dissolvable drug membrane is 0.01mm-1 mm, an area thereof is 0.2 cm²-25 cm²; wherein 1 cm² of the sterile and instantly dissolvable drug membrane is dissolved in 1 ml of cell culture fluid within 5 min, preferably 3 min, more preferably 100 sec.

13. The method, as recited in claim 11, wherein the sterile and instantly dissolvable drug membrane comprises the antineoplastic drug with an amount of 0.0001 wt %-30 wt %, a membrane forming material with an amount of 50 wt %-98 wt %, and a plasticizer with an amount of 1 wt %-20wt %.

14. The method, as recited in claim 12, wherein the sterile and instantly dissolvable drug membrane comprises the antineoplastic drug with an amount of 0.0001 wt %-30wt %, a membrane forming material with an amount of 50 wt %-98 wt %, and a plasticizer with an amount of 1 wt %-20 wt %.

15. The method, as recited in claim 11, wherein the antineoplastic drug is selected from a group consisting of fluorouracil, tegafur, tegadifur, doxifluridine, carmo fur, methotrexate, busulfan, doxorubicin, daunorubicin, idarubicin, epirubicin, pirarubicin, mitoxantrone, actinomycin D, bleomycin, pingyangmycin, mitomycin, mithramycin, olivomycin, streptozocin, mechlorethamine, chlorambucil, melphalan, cyclophosphamide, ifosfamide, thiotepa, carmustine, lomustine, cytarabine, floxuridine, cyclocylidine, chlorine cyclocylidine, fludarabine, gemcitabine, docetaxel, vinorelbine, vindesine, vincristine, paclitaxel, camptothecin, hydroxy camptothecin, cisplatin, oxaliplatin, carboplatin, nedaplatin, lobaplatin, platinum heptyl, rituximab, ibritumomab, cetuximab, bevacizumab, interleukin-2, leuprorelin acetate, goserelin acetate, anastrozole, letrozole, aminoglutethimide, formestane, exemestane, teniposide, etoposide, pentostatin, irinotecan, sorafenib, capecitabine, decitabine, pemetrexed disodium, procarbazine, harringtonine, thalidomide, dacarbazine, tamoxifen, temozolomide, topotecan, gefitinib, erlotinib, flutamide, nilutamide, bicalutamide, exemestane, arsenite and indirubin.

16. The method, as recited in claim 12, wherein the antineoplastic drug is selected from a group consisting of fluorouracil, tegafur, tegadifur, doxifluridine, carmo fur, methotrexate, busulfan, doxorubicin, daunorubicin, idarubicin, epirubicin, pirarubicin, mitoxantrone, actinomycin D, bleomycin, pingyangmycin, mitomycin, mithramycin, olivomycin, streptozocin, mechlorethamine, chlorambucil, melphalan, cyclophosphamide, ifosfamide, thiotepa, carmustine, lomustine, cytarabine, floxuridine, cyclocylidine, chlorine cyclocylidine, fludarabine, gemcitabine, docetaxel, vinorelbine, vindesine, vincristine, paclitaxel, camptothecin, hydroxy camptothecin, cisplatin, oxaliplatin, carboplatin, nedaplatin, lobaplatin, platinum heptyl, rituximab, ibritumomab, cetuximab, bevacizumab, interleukin-2, leuprorelin acetate, goserelin acetate, anastrozole, letrozole, aminoglutethimide, formestane, exemestane, teniposide, etoposide, pentostatin, irinotecan, sorafenib, capecitabine, decitabine, pemetrexed disodium, procarbazine, harringtonine, thalidomide, dacarbazine, tamoxifen, temozolomide, topotecan, gefitinib, erlotinib, flutamide, nilutamide, bicalutamide, exemestane, arsenite and indirubin.

17. The method, as recited in claim 13, wherein the antineoplastic drug is selected from a group consisting of fluorouracil, tegafur, tegadifur, doxifluridine, carmo fur, methotrexate, busulfan, doxorubicin, daunorubicin, idarubicin, epirubicin, pirarubicin, mitoxantrone, actinomycin D, bleomycin, pingyangmycin, mitomycin, mithramycin, olivomycin, streptozocin, mechlorethamine, chlorambucil, melphalan, cyclophosphamide, ifosfamide, thiotepa, carmustine, lomustine, cytarabine, floxuridine, cyclocylidine, chlorine cyclocylidine, fludarabine, gemcitabine, docetaxel, vinorelbine, vindesine, vincristine, paclitaxel, camptothecin, hydroxy camptothecin, cisplatin, oxaliplatin, carboplatin, nedaplatin, lobaplatin, platinum heptyl, rituximab, ibritumomab, cetuximab, bevacizumab, interleukin-2, leuprorelin acetate, goserelin acetate, anastrozole, letrozole, aminoglutethimide, formestane, exemestane, teniposide, etoposide, pentostatin, irinotecan, sorafenib, capecitabine, decitabine, pemetrexed disodium, procarbazine, harringtonine, thalidomide, dacarbazine, tamoxifen, temozolomide, topotecan, gefitinib, erlotinib, flutamide, nilutamide, bicalutamide, exemestane, arsenite and indirubin.

18. The method, as recited in claim 14, wherein the antineoplastic drug is selected from a group consisting of fluorouracil, tegafur, tegadifur, doxifluridine, carmo fur, methotrexate, busulfan, doxorubicin, daunorubicin, idarubicin, epirubicin, pirarubicin, mitoxantrone, actinomycin D, bleomycin, pingyangmycin, mitomycin, mithramycin, olivomycin, streptozocin, mechlorethamine, chlorambucil, melphalan, cyclophosphamide, ifosfamide, thiotepa, carmustine, lomustine, cytarabine, floxuridine, cyclocylidine, chlorine cyclocylidine, fludarabine, gemcitabine, docetaxel, vinorelbine, vindesine, vincristine, paclitaxel, camptothecin, hydroxy camptothecin, cisplatin, oxaliplatin, carboplatin, nedaplatin, lobaplatin, platinum heptyl, rituximab, ibritumomab, cetuximab, bevacizumab, interleukin-2, leuprorelin acetate, goserelin acetate, anastrozole, letrozole, aminoglutethimide, formestane, exemestane, teniposide, etoposide, pentostatin, irinotecan, sorafenib, capecitabine, decitabine, pemetrexed disodium, procarbazine, harringtonine, thalidomide, dacarbazine, tamoxifen, temozolomide, topotecan, gefitinib, erlotinib, flutamide, nilutamide, bicalutamide, exemestane, arsenite and indirubin.

19. A sterile and instantly dissolvable drug membrane for testing drug sensitivity of antineoplastic drugs, comprising: an antineoplastic drug with an amount of 0.0001 wt %-30wt %, a membrane forming material with an amount of 50wt %-98 wt %, and a plasticizer with an amount of 1 wt %-20 wt %; wherein the membrane forming material is polyvinyl alcohol, hydroxypropyl methyl cellulose, hydroxyethyl propyl cellulose, sodium carboxymethyl cellulose, or polyvinylpyrrolidone; the plasticizer is glycerol, polyethylene glycol, ethylene glycol, propylene glycol, sorbitol, or triethyl citrate.

20. The sterile and instantly dissolvable drug membrane, as recited in claim 19, wherein a thickness of the sterile and instantly dissolvable drug membrane is 0.01 mm-1 mm, an area thereof is 0.2 cm²-25 cm²; wherein 1 cm² of the sterile and instantly dissolvable drug membrane is dissolved in 1 ml of cell culture fluid within 5min, preferably 3 min, more preferably 100 sec.

21. The sterile and instantly dissolvable drug membrane, as recited in claim 19, wherein the antineoplastic drug is selected from a group consisting of fluorouracil, tegafur, tegadifur, doxifluridine, carmo fur, methotrexate, busulfan, doxorubicin, daunorubicin, idarubicin, epirubicin, pirarubicin, mitoxantrone, actinomycin D, bleomycin, pingyangmycin, mitomycin, mithramycin, olivomycin, streptozocin, mechlorethamine, chlorambucil, melphalan, cyclophosphamide, ifosfamide, thiotepa, carmustine, lomustine, cytarabine, floxuridine, cyclocylidine, chlorine cyclocylidine, fludarabine, gemcitabine, docetaxel, vinorelbine, vindesine, vincristine, paclitaxel, camptothecin, hydroxy camptothecin, cisplatin, oxaliplatin, carboplatin, nedaplatin, lobaplatin, platinum heptyl, rituximab, ibritumomab, cetuximab, bevacizumab, interleukin-2, leuprorelin acetate, goserelin acetate, anastrozole, letrozole, aminoglutethimide, formestane, exemestane, teniposide, etoposide, pentostatin, irinotecan, sorafenib, capecitabine, decitabine, pemetrexed disodium, procarbazine, harringtonine, thalidomide, dacarbazine, tamoxifen, temozolomide, topotecan, gefitinib, erlotinib, flutamide, nilutamide, bicalutamide, exemestane, arsenite and indirubin.

22. The sterile and instantly dissolvable drug membrane, as recited in claim 20, wherein the antineoplastic drug is selected from a group consisting of fluorouracil, tegafur, tegadifur, doxifluridine, carmofur, methotrexate, busulfan, doxorubicin, daunorubicin, idarubicin, epirubicin, pirarubicin, mitoxantrone, actinomycin D, bleomycin, pingyangmycin, mitomycin, mithramycin, olivomycin, streptozocin, mechlorethamine, chlorambucil, melphalan, cyclophosphamide, ifosfamide, thiotepa, carmustine, lomustine, cytarabine, floxuridine, cyclocylidine, chlorine cyclocylidine, fludarabine, gemcitabine, docetaxel, vinorelbine, vindesine, vincristine, paclitaxel, camptothecin, hydroxy camptothecin, cisplatin, oxaliplatin, carboplatin, nedaplatin, lobaplatin, platinum heptyl, rituximab, ibritumomab, cetuximab, bevacizumab, interleukin-2, leuprorelin acetate, goserelin acetate, anastrozole, letrozole, aminoglutethimide, formestane, exemestane, teniposide, etoposide, pentostatin, irinotecan, sorafenib, capecitabine, decitabine, pemetrexed disodium, procarbazine, harringtonine, thalidomide, dacarbazine, tamoxifen, temozolomide, topotecan, gefitinib, erlotinib, flutamide, nilutamide, bicalutamide, exemestane, arsenite and indirubin.

23. A method for preparing a sterile and instantly dissolvable drug membrane as recited in claim 19, comprising steps of:
(1) adding a membrane forming material into water, and stirring or heating for dissolving;
(2) adding a plasticizer and an antineoplastic drug, stirring for thoroughly dissolving, and degassing with heating, staying, or ultrasound;
(3) coating a solution obtained above on a membrane producer, and drying with hot wind or cold wind;
(4) stripping and measuring contents thereof, dividing according to a measuring result for obtaining a pre-determined dosage; and
(5) after dividing, sterilizing with irradiation sterilization or epoxyethane sterilization, preferably the irradiation sterilization.

24. A method for testing drug sensitivity of antineoplastic drugs, comprising applying a sterile and instantly dissolvable drug membrane as recited in claim 19.

25. The method, as recited in claim 24, wherein the sterile and instantly dissolvable drug membrane is directly used in a tumor cell drug sensitivity test, or the sterile and instantly dissolvable drug membrane is dissolved and then activated by adding drug metabolic enzyme, for being used in the tumor cell drug sensitivity test.
